(19) United States
(12) Patent Application Publication
(10) Pub. No.: US 2020/0216888 A1
(43) Pub. Date: Jul. 9, 2020

(54) METHOD FOR INCREASING ACCURACY OF ANALYSIS BY REMOVING PRIMER SEQUENCE IN AMPLICON-BASED NEXT-GENERATION SEQUENCING

(71) Applicant: NGENEBIO, Seoul (KR)

(72) Inventors: Chang Seon LEE, Incheon (KR); Chang Bum HONG, Gyeonggi-do (KR); Ensel OH, Seoul (KR); Kwang Joong KIM, Sejong (KR)

(21) Appl. No.: 16/637,880

(22) PCT Filed: Aug. 9, 2018
(86) PCT No.: PCT/KR18/09088

§ 371 Date: Feb. 10, 2020

(30) Foreign Application Priority Data
Aug. 10, 2017 (KR) 10-2017-0101540

(51) Int. Cl. C12Q 1/6869; G16B 25/00; G16B 40/30; C12Q 1/6853

(52) U.S. Cl. CPC C12Q 1/6869; C12Q 1/6853; G16B 40/30; G16B 25/00

(57) ABSTRACT

The present invention relates to a method for increasing the efficiency of read data analysis by removing primer sequence information present in a read obtained through next-generation sequencing (NGS) and, more specifically, to a method for matching information of a read and a designed primer to various reference values in several steps so as to determine primer sequence information within a read, and then precisely removing only a primer sequence so as to increase the efficiency of read data analysis. The method for increasing the efficiency of read data analysis in a primer removal-based NGS, according to the present invention, has a rapid data analysis speed and can precisely remove only a primer sequence, thereby being useful for increasing the efficiency and accuracy of read data analysis.

### TECHNICAL FIELD

The present invention relates to a method for increasing the efficiency of read data analysis by removing primer sequence information present in a read obtained through next-generation sequencing (NGS) and, more specifically, to a method for increasing the efficiency of read data analysis by matching a read and designed primer information to various reference values through several steps to determine the primer sequence information within the read, and then precisely removing only the primer sequence.

### BACKGROUND ART

Over the past decade, next-generation sequencing (NGS) has attracted much attention in the field of genetic analysis. Unlike conventional methods, next-generation sequencing is a technology that can produce large amounts of data quickly and thus dramatically reduce the time and cost required to decipher individual genomes. With regard to next-generation sequencing, sequencing platforms have been gradually developed, and the cost of analysis has been gradually reduced, and thus next-generation sequencing has been used to successfully find the genes causing Mendelian genetic diseases, rare diseases and cancer (Buermans H P J et al., Biochim. Biophys. Acta. 1842 (10): 1932-41, 2014). In accordance with next-generation sequencing, DNA is extracted from a sample and subjected to mechanical fragmentation, and then a library having a specific size is produced and used for sequencing. Next-generation sequencing involves repeating four types of complementary nucleotide binding and separation reactions with one base unit using a large-scale sequencing apparatus to produce initial sequencing data, and performing analysis steps using bioinformatics such as trimming initial data, mapping, identifying genomic variations, and annotating variation information to discover genomic variations that affect or have a strong possibly of affecting diseases and various biophenotypes, thereby contributing to the creation of new added value through the development and industrialization of innovative therapeutics.

Among these next-generation sequencing methods, an amplicon-based NGS method includes designing a primer that can amplify a target gene to produce a variety of short-length reads, and then aligning and analyzing the same. A representative technique is an emulsion PCR method, and devices based thereon include Roche's 454 platform, Thermo FIsher's SOLiD platform, Ion Torrent platform and the like. The amplicon-based NGS method has advantages of lower library complexity, but higher analysis speed compared to a probe-based hybridization method (Sara Goodwin et al., Nature Reviews Genetics, Vol 17: 333-51, 2016).

Amplicon-type NGS data has a primer sequence present in the front sequence of the read. This primer sequence is designed with the same sequence as the standard sequence. In the case where part of the primer sequence overlaps the part where the variation in the sample exists, when the variation is homo, the primer is the same as the standard sequence, and the part where the variation exists appears to be hetero. In the case of a hetero variant, determination as being hetero is difficult due to variant allele frequency lower than the original level. That is, the primer sequence may be different from the sequence in the actual sample because the primer sequence is produced based on the reference gene. Thus, when the primer is not removed, the sequence of the primer and the sequence of the actual sample having the variation are present in a mixed form, thus affecting the allele frequency. Therefore, when this part is used for analysis, without removing it, there is a problem in that it acts as a false positive in variation detection.

There are various programs for solving the above problems. Conventional programs have a disadvantage in that the primer removal accuracy is low because of use only one reference value and it takes a long time to detect and remove primer sequences.

Accordingly, as a result of extensive effort to solve the above problems, the present inventors found that, when comparing and analyzing read sequence information with primer sequence information using various methods and various reference values, the primer sequence can be accurately determined, sensitivity and accuracy can be maintained, and consumption of time and expenses can be greatly reduced. Based on these findings, the present invention has been completed.

### DISCLOSURE

### Technical Problem

It is one object of the present invention to provide a method of increasing accuracy of analysis of read data through primer removal in next-generation sequencing (NGS).

It is another object of the present invention to provide a computer system including a computer-readable medium encoded with a plurality of instructions for controlling a computing system to perform primer sequence removal in amplicon-based next-generation sequencing (NGS).

### Technical Solution

In accordance with one aspect of the present invention, the above and other objects can be accomplished by the provision of a method of increasing the accuracy of analysis of read data through primer removal in next-generation sequencing (NGS), including: (a) acquiring a read through amplicon-based next-generation sequencing; (b) analyzing a primer sequence and the read sequence to determine the primer sequence in the read sequence; and (c) removing the determined primer sequence.

In accordance with another aspect of the present invention, provided is a computer system including a computer-readable medium encoded with a plurality of instructions for controlling a computing system to perform primer sequence removal in amplicon-based next-generation sequencing (NGS),
wherein the computer system includes: (a) acquiring a read through amplicon-based next-generation sequencing; (b) analyzing a primer sequence and the read sequence to determine the primer sequence in the read sequence; and (c) removing the determined primer sequence.

### DESCRIPTION OF DRAWINGS

FIG. 1 is a schematic view illustrating a method of removing a primer according to the present invention.

FIG. 2A is a schematic diagram showing a part of the alignment of amplicon designed in the BRCA2 gene according to an embodiment of the present invention, and FIG. 2B shows an actual sequence of a part of the read of FIG. 2A.

FIG. 3 shows a combination of amplicon primers according to an embodiment of the present invention.

FIG. 4 is a graph showing comparison in a primer removal completion time between the method according to the present invention and a conventional well-known program.

FIG. 5 is a graph showing the number of reads that can be used for analysis after completion of primer removal in the method according to the present invention and the conventional well-known program.

FIG. 6 shows the result of analysis of accuracy when aligning the reads after the primer removal in the method according to the present invention and the conventional well-known program.

### BEST MODE

Unless defined otherwise, all technical and scientific terms used herein have the same meanings as appreciated by those skilled in the field to which the present invention pertains. In general, the nomenclature used herein is well-known in the art and is ordinarily used.

As used herein, the term “next-generation sequencing” or “NGS” refers to a sequencing method that determines the nucleotide sequence of one of proxies expanded with clones for an individual nucleic acid molecule in an individual nucleic acid molecule mode (e.g., in single-molecule sequencing) or in high-speed bulk mode (e.g., when sequencing 10, 100, 1000 or more molecules simultaneously). In one embodiment, the relative abundance of nucleic acid species in the library can be estimated by measuring, in the data generated by the sequencing experiments, the relative number of occurrences of cognate sequences thereof. Next-generation sequencing methods are known in the art and are described, for example, in [Metzker, M. (2010) Nature Biotechnology Reviews 11:31-46]. Next-generation sequencing can detect variants present in less than 5% of nucleic acids in a sample.

The next-generation sequencing process in the present invention can be divided into the following three steps.

(1) Amplification of Target

Next-generation sequencing can be used to sequence the whole genome, to sequence only exome regions (targeted sequencing), or to sequence only specific genes in order to find genes causative of diseases. Sequencing only exome regions or specific target genes is advantageous in terms of cost or efficiency. In addition, since variations of genes are often directly caused by diseases such as cancer, detecting the change in the nucleotide sequence in the exome region or the target gene may be effective in finding genes causative of diseases. In order to sequence only exomes or target genes, a library capable of amplifying only the exomes or target genes is required.

In order to amplify only target genes, primers specific to the certain target genes may be used.

(2) Large-Capacity Parallel DNA Sequencing

Next-generation sequencing (NGS) has advantages of simultaneously identifying a greater amount of sequences more quickly at once than conventional capillary sequencing, and of omitting a process of amplifying the sample, thus avoiding experimental error occurring in this process.

NGS systems produced by three companies are mainly used. 454 GS FLX of Roche AG launched in 2004 was the first NGS instrument capable of performing sequencing using pyrosequencing and emulsion polymerase chain reactions and determining specific bases depending on the intensity of light emitted during the final stage of the experiment. When operated for 7 hours, the 454 GS FLX can identify a sequence of about 100 Mb, which is much higher than a conventional ABI 3730 device, which can identify a sequence of 440 kb within the same time.

The Illumina genome analyzer produced by Illumina, Inc. is based on the concept of sequencing by synthesis. After attaching single-stranded DNA fragments onto a glass plate, the fragments are polymerized and clustered. During this process, sequence analysis is performed while determining the type of bases attached to the DNA fragments to be tested. After operation for about four days, about 40 to 50 million fragments having a base length of 32 to 40 are produced.

The SOLiD (sequencing by oligo ligation) apparatus produced by Life Technologies Inc. is designed to perform sequencing using an emulsifier-polymerase chain reaction after attaching a DNA fragment to be tested to a 1 μm magnetic bead. Sequencing is carried out by repeatedly attaching 8-mer fragments to each other. The bases used for actual sequencing are positioned at the 4^{th} and 5^{th} 8-mer fragments. A fluorescent material is linked to the remainder behind them to mark the base that complementarily binds to the DNA fragment to be tested. By attaching all 8-mers five times in one binding cycle and performing the same operation five times, a sequence of DNA fragments consisting of a total of 25 bases can be identified. The SOLiD instrument is characterized by sequencing using two-base encoding. This method identifies the same region through double sequencing when determining the sequence of one base. Sequencing is performed while shifting the sequence by one base in one binding cycle toward the adaptor attached to the magnetic bead. This process has the advantage of eliminating errors that occur in sequencing experiments.

(3) Analysis of Base Sequence Data

In order to find genes causative of diseases, it is necessary to investigate what changes have been made from the original gene sequence. Thus, an operation of comparing nucleotide data (sequence reads) of an individual (patient) with the reference genome is performed. This operation is called mapping. Differences between the individual sequence and the reference sequence are identified through mapping, appropriate selection criteria are set based on the differences, and only reliable sequence variant information is extracted (variant calling). This variation information is structural variation (SV) that includes single nucleotide variation (SNV), short indel, copy number variation (CNV), fusion genes and the like. Then, the nucleotide variation information is compared with the existing database to determine whether it is a known or newly discovered variation. Also, whether or not the variation will result in a change in amino acids and how it affects protein structure is predicted. This process is called “annotation”. Information associated with extracted single-nucleotide sequence variations and short indel may be listed in the database so as to improve the quality of the information, or research to find variations causative of diseases can be conducted through studies integrated with the genome wild association study (GWAS).

However, the conventional method has a disadvantage in that it takes a long time to remove the primer information from the amplicon-type read. Thus, according to the present invention, a method of determining the primer sequence information with high accuracy and removing the same has been developed.

As used herein, the term “acquire” or “acquiring” refers to possessing a physical entity or value, such as a numerical value, by “directly acquiring” or “indirectly acquiring” a physical entity or value. “Indirectly acquiring” means performing a process to acquire a physical entity or value (e.g., performing a synthetic or analytical method). “Indirectly acquiring” refers to receiving a physical entity or value from another party or source (e.g., a third-party laboratory that directly acquired the physical entity or value).

Indirectly acquiring a physical entity involves performing a process involving a physical change from a physical material, for example, a starting material. Representative changes include performing chemical reactions involving forming physical entities from two or more starting materials, shearing or fragmenting materials, separating or purifying materials, combining two or more separate entities into a mixture, and breaking or forming a covalent or non-covalent bond. Indirectly acquiring a value includes performing a treatment involving a physical change from a sample or other material, for example, performing an analytical process involving a physical change from a material, for example, a sample, analyte or reagent (often referred to herein as “physical analysis”), performing an analytical method, e.g., a method including one or more of the following: separating or purifying a material, such as an analyte or fragment or other derivative thereof, from another material; combining an analyte or fragment or other derivative thereof with another material, such as a buffer, solvent or reactant; or changing the structure of an analyte or fragment or other derivative thereof, for example, by breaking or forming a covalent or non-covalent bond between the first and second atoms of the analyte; or changing the structure of a reagent or fragment or other derivative thereof, for example by breaking or forming a covalent or non-covalent bond between the first and second atoms of the reagent.

As used herein, the term “acquiring a sequence” or “acquiring a read” refers to possessing a nucleotide sequence or amino acid sequence by “directly acquiring” or “indirectly acquiring” the sequence or read. “Directly acquiring” a sequence or read refers to performing a process for acquiring the sequence (e.g., performing a synthetic or analytical method), for example, performing a sequencing method (e.g., a next-generation sequencing (NGS) method). “Indirectly acquiring” a sequence or read refers to receiving a sequence from another party or source (e.g., a third-party laboratory that directly acquired the sequence) or receiving information or knowledge of the sequence. The acquired sequence or read need not be a complete sequence, and acquiring information or knowledge to identify one or more of the alterations disclosed herein, for example, sequencing of at least one nucleotide or presence in a subject, constitutes acquiring a sequence.

Directly acquiring a sequence or read includes performing a process involving a physical change from a physical material, e.g., a starting material, such as a tissue or cell sample, e.g., a biopsy or an isolated nucleic acid (e.g., DNA or RNA) sample. Representative changes include shearing or fragmenting two or more materials, for example, starting materials, such as producing physical entities from genomic DNA fragments (e.g., separating nucleic acid samples from tissue); performing a chemical reaction including combining two or more separate entities into a mixture, and breaking or forming a covalent or non-covalent bond. Directly acquiring a value includes performing a process involving a physical change from the sample or other material as described above.

As used herein, the term “nucleic acid” or “polynucleotide” refers to a single- or double-stranded deoxyribonucleic acid (DNA) or ribonucleic acid (RNA) and polymers thereof. Unless specifically limited otherwise, the term includes nucleic acids containing known analogues of natural nucleotides that have binding properties similar to those of the reference nucleic acid and are metabolized in a manner similar to natural nucleotides. Unless otherwise stated, certain nucleic acid sequences also include not only clearly disclosed sequences but also implicitly conservatively modified variants thereof (e.g., degenerate codon substitutions), alleles, orthologs, SNPs and complementary sequences. Specifically, degenerate codon substitutions can be carried out by forming a sequence in which position 3 of one or more selected codons (or all codons) is substituted with a mixed base and/or a deoxyinosine residue (Batzer et al., Nucleic Acid Res. 19:5081 (1991); Ohtsuka et al., J. Biol. Chem. 260:2605-2608 (1985); and Rossolini et al., MoI. Cell. Probes 8:91-98 (1994)). The term “nucleic acid” is used interchangeably with a gene, cDNA, mRNA, small non-coding RNA, micro RNA (miRNA), Piwi-interacting RNA and short hairpin RNA (shRNA) encoded by a gene or locus.

As herein used, the term “reference error value (%)” means a number used for analysis between the primer sequence and the read sequence. For example, a primer sequence matching the read sequence with an error greater than the reference error value is classified as an error, and a primer sequence matching the read sequence with an error lower than the reference error value is classified as normal.

As used herein, the term “paired-end read” refers to two ends of the same DNA molecule. When one end is sequenced and then turned over and the other end is sequenced, these two ends, the base sequence of which is identified, are called “paired-end reads”. For example, Illumina sequencing generates a read of about 500 bps and reads a nucleotide sequence 75 bps long at each end of the read. At this time, the reading directions of the two reads (the first read and the second read) are 3′ and 5′, which are opposite to each other, respectively, and mutually become paired-end reads.

As used herein, the terms “first read”, “second read”, “pair 1”, and “pair 2” refer to a first read in the 5′ direction (pair 1) and a second read (pair 2) in the 3′ direction, acquired through paired-end read sequencing.

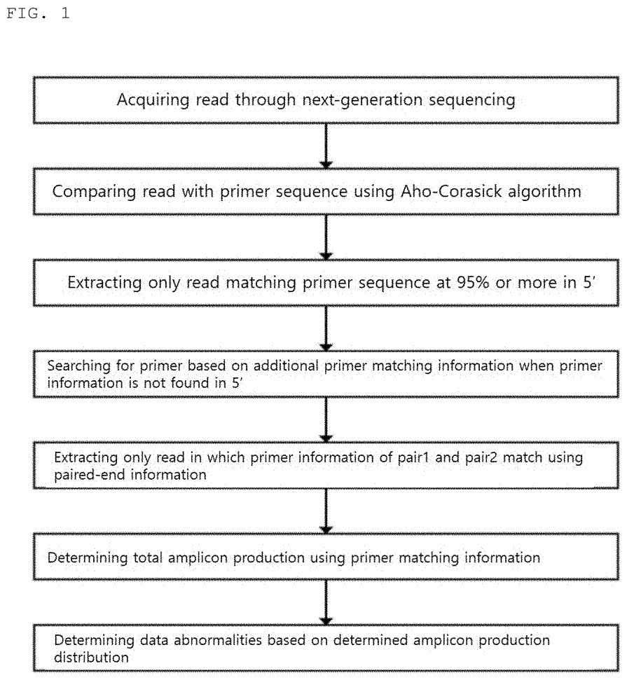

In the present invention, whether or not the primer sequence information inside the read sequence can be removed using various reference values and various methods is determined (FIG. 1).

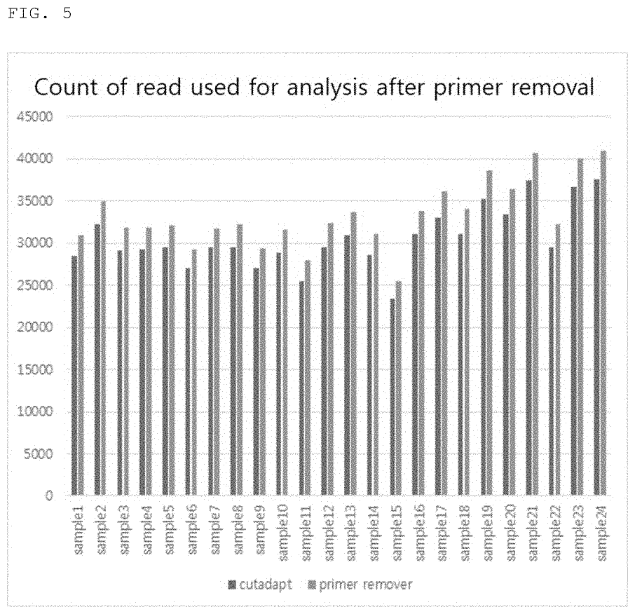

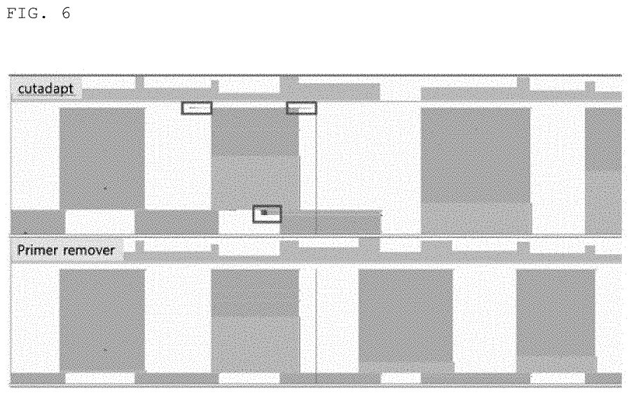

That is, in an embodiment of the present invention, reads for BRCA 1 and 2 genes are acquired through amplicon-based NGS, previously designed primer sequence information is matched with the read sequence to extract a 100% matched read sequence, two kinds of sequences are re-matched at a reference error value of 5% to extract a 95% matched read sequence, the primer sequence information of the read is determined based on the primer sequence information inside the read in the unextracted read sequence to determine primer sequence information of the acquired read, and the primer sequence was removed from the read. The time (FIG. 4), the number of remaining reads (FIG. 5), and the accuracy thereof (FIG. 6) were compared. The results showed that the method of the present invention is excellent in all respects compared to conventional well-known programs.

In one aspect, the present invention is directed to a method of increasing accuracy of analysis of read data through primer removal in amplicon-based next-generation sequencing (NGS), including: (a) acquiring a read through amplicon-based next-generation sequencing; (b) analyzing a primer sequence and the read sequence to determine the primer sequence in the read sequence; and (c) removing the determined primer sequence.

In the present invention, the read of step (a) may be saved in a fastq file format, but is not limited thereto.

In the present invention, step (b) includes: (i) extracting a read sequence completely matching a primer sequence from the read sequence; (ii) extracting a read sequence matching the primer sequence at a reference error value (%) from the read sequence not extracted in step (i); and (iii) determining primer sequence information of the read based on primer sequence information inside the read from the primer sequence and the read sequence not extracted in step (ii).

In the present invention, “completely matching” in step (i) may mean that the primer sequence information 100% matches the read sequence information, wherein the matching is carried out using the Aho-Corasick algorithm, but is not limited thereto.

In the present invention, the read sequence of step (i) may be characterized in that the 5′ portion is removed in an amount of 1 to 65% of the entire length of the primer, preferably 20% thereof, but is not limited thereto.

In the present invention, the read sequence in step (i) may be characterized in that the 5′ portion is removed in a length of 1 bp to 13 bp, preferably 5 bp, when the entire length of the primer is 21 to 36 bp, but is not limited thereto.

In the present invention, the sequence comparison in step (i) may be characterized by comparing the primer sequence with 20 bp to 70 bp of the 5′ portion of the read sequence, preferably 50 bp thereof, but is not limited thereto.

In the present invention, the sequence comparison in step (i) may be characterized by comparing the primer sequence with 10 to 50% of the 5′ portion of the read sequence, preferably 30% thereof, but is not limited thereto.

In the present invention, the reference error value (%) in step (ii) may be used without limitation as long as it is a value that can accurately determine the primer sequence in the read sequence, and the reference error value (%) in step (ii) is preferably 0.1% to 10%, and most preferably 5%, but is not limited thereto.

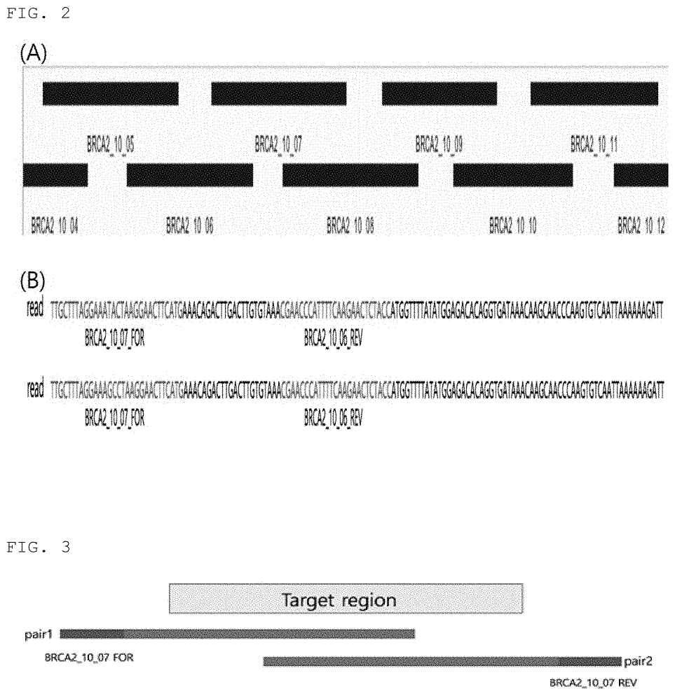

In the present invention, the primer sequence information inside the read in step (iii) may be information corresponding to the primer sequence of another read present inside the read sequence. That is, in the present invention, since reads are designed to overlap one another, in one read, sequence information of the part corresponding to the primer of another read is present (FIG. 2).

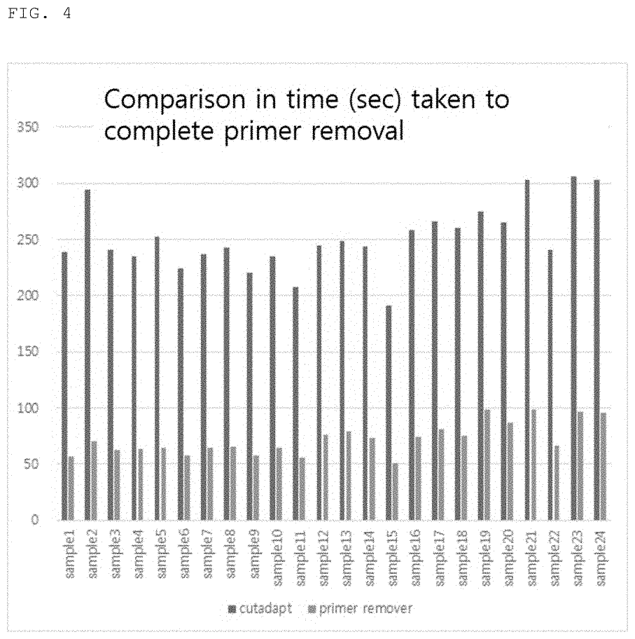

In the present invention, determining the primer sequence in step (b) may include determining and saving read information and primer information when the primers of the same read are forward (5′) and reverse (3′) primers, respectively, and correspond (match) to each other, based on the result of sequencing of the first and second reads (FIG. 3).

In the present invention, the method may further include determining and reporting the ratio of the read in which the primer sequence is determined from the entire read sequence in step (b) to the read in which the primer sequence is not determined therefrom.

In the present invention, when the next-generation sequencing method is based on amplicon, the method may further include reporting the presence or absence of data abnormalities through an amplicon production result.

In the present invention, the amplicon production result may be obtained by comparing the amplicon production result predicted based on the primer matching result of an experimental sample with the amplicon production result of the experimental sample compared to an actual control sample.

In another aspect, the present invention is directed to a computer system including a computer-readable medium encoded with a plurality of instructions for controlling a computing system to perform primer sequence removal in next-generation sequencing (NGS), wherein the computer system includes: (a) acquiring a read through amplicon-based next-generation sequencing; (b) analyzing a primer sequence and the read sequence to determine the primer sequence in the read sequence; and (c) removing the determined primer sequence.

In the present invention, step (b) may include: (i) extracting a read sequence completely matching a primer sequence from the read sequence; (ii) extracting a read sequence matching the primer sequence at a reference error value (%) from the read sequence not extracted in step (i); and (iii) determining primer sequence information of the read based on primer sequence information inside the read from the primer sequence and the read sequence not extracted in step (ii).

Hereinafter, the present invention will be described in more detail with reference to examples. However, it will be obvious to those skilled in the art that these examples are provided only for illustration of the present invention and should not be construed as limiting the scope of the present invention.

### EXAMPLE 1

### Acquisition of NGS-Based Read

Amplicon-based NGS was performed with a standard material having variation in the BRCA gene to acquire the number of reads for the BRACA gene in each sample shown in Table 1 below.

TABLE 1
BRCA read raw count

| Sample # | Read count |
|---|---|
| Sample 1 | 38329 |
| Sample 2 | 42871 |
| Sample 3 | 38410 |
| Sample 4 | 38881 |
| Sample 5 | 40867 |
| Sample 6 | 36741 |
| Sample 7 | 39031 |
| Sample 8 | 39541 |
| Sample 9 | 36601 |
| Sample 10 | 39747 |
| Sample 11 | 35189 |
| Sample 12 | 40638 |
| Sample 13 | 41649 |
| Sample 14 | 40010 |
| Sample 15 | 31768 |
| Sample 16 | 41566 |
| Sample 17 | 43909 |
| Sample 18 | 41652 |
| Sample 19 | 46255 |
| Sample 20 | 43950 |
| Sample 21 | 50263 |
| Sample 22 | 40038 |
| Sample 23 | 49956 |
| Sample 24 | 49082 |

### EXAMPLE 2

### Comparison Between Primer Sequence Information and Read Sequence With Aho-Corasick Algorithm

5 bp of the 5′ portion was removed from the acquired 30,000 read sequences, and the designed primer sequence information was compared with the reads based on the Aho-Corasick algorithm to extract a 100%-matched read (primer sequence is determined) of each sample, shown in Table 2 below.

TABLE 2
Results of primary analysis of BRCA read - Aho-Corasick

| Sample # | Read count |
|---|---|
| Sample 1 | 36310 |
| Sample 2 | 40414 |
| Sample 3 | 36552 |
| Sample 4 | 36807 |
| Sample 5 | 38281 |
| Sample 6 | 34406 |
| Sample 7 | 36934 |
| Sample 8 | 37460 |
| Sample 9 | 34568 |
| Sample 10 | 37438 |
| Sample 11 | 33268 |
| Sample 12 | 38278 |
| Sample 13 | 38973 |
| Sample 14 | 37417 |
| Sample 15 | 30169 |
| Sample 16 | 39332 |
| Sample 17 | 41585 |
| Sample 18 | 39466 |
| Sample 19 | 43909 |
| Sample 20 | 41498 |
| Sample 21 | 47681 |
| Sample 22 | 37799 |
| Sample 23 | 47449 |
| Sample 24 | 46518 |

### EXAMPLE 3

### Comparison of Primer Sequence Information With Read Sequence Based on Reference Error Value (%)

The read that did not match 100% and was thus not extracted in Example 2 was matched with the primer sequence at a reference error value of 5%, and a 95% matched read (primer sequence is determined) of each sample was extracted, as shown in Table 3 below.

TABLE 3
Results of primary analysis of BRCA read - reference error value

| Sample # | Read count |
|---|---|
| Sample 1 | 248 |
| Sample 2 | 285 |
| Sample 3 | 224 |
| Sample 4 | 259 |
| Sample 5 | 274 |
| Sample 6 | 232 |
| Sample 7 | 238 |
| Sample 8 | 277 |
| Sample 9 | 219 |
| Sample 10 | 228 |
| Sample 11 | 221 |
| Sample 12 | 238 |
| Sample 13 | 264 |
| Sample 14 | 248 |
| Sample 15 | 210 |
| Sample 16 | 222 |
| Sample 17 | 291 |
| Sample 18 | 284 |
| Sample 19 | 291 |
| Sample 20 | 304 |
| Sample 21 | 311 |
| Sample 22 | 242 |
| Sample 23 | 296 |
| Sample 24 | 299 |

### EXAMPLE 4

### Determination of Primer Sequence Based on Primer Sequence Information in Read

5′ primer sequence information of each read was determined, based on information of another read present in the read (FIGS. 2A and 2B), from the read not extracted in Example 3, as shown in Table 4 below.

TABLE 4
Results of tertiary analysis of BRCA
read - internal primer information

| Sample # | Read count |
|---|---|
| Sample 1 | 41 |
| Sample 2 | 53 |
| Sample 3 | 37 |
| Sample 4 | 39 |
| Sample 5 | 51 |
| Sample 6 | 35 |
| Sample 7 | 37 |
| Sample 8 | 55 |
| Sample 9 | 43 |
| Sample 10 | 43 |
| Sample 11 | 35 |
| Sample 12 | 43 |
| Sample 13 | 42 |
| Sample 14 | 34 |
| Sample 15 | 19 |
| Sample 16 | 42 |
| Sample 17 | 48 |
| Sample 18 | 43 |
| Sample 19 | 52 |
| Sample 20 | 42 |
| Sample 21 | 51 |
| Sample 22 | 38 |
| Sample 23 | 40 |
| Sample 24 | 52 |

### EXAMPLE 5

### Final Determination of Primer Sequences and Removal of Primer Sequence

When the primers in the first and second reads were forward (5′) and reverse (3′) primers respectively and are matched, based on the primer sequence information determined in Examples 2 to 4, read information and primer information were determined and saved, and then primer sequence information was removed.

TABLE 5
Determination of primer pairs

| | Pair1 | Pair2 | save |
|---|---|---|---|
| Read1 | BRCA2_10_07_FOR | BRCA2_10_07_REV | ◯ |
| Read2 | BRCA2_10_07_FOR | BRCA2_10_09_REV | X |
| Read3 | BRCA2_10_07_REV | BRCA2_10_07_FOR | ◯ |

### EXAMPLE 6

### Comparison Between Method of Present Invention and Well-Known Program

6-1. Comparison in Primer Removal Rate

With regard to 24 samples (each having 30,000 raw reads), the times taken until the primer was completely removed were compared between the method of the present invention and a well-known program (cutadapt, https://github.com/marcelm/cutadapt). The result showed that the method of the present invention completed primer removal much quickly (Table 6, FIG. 4). That is, the method of the present invention took about 72 seconds on average to complete the analysis, which was 2.6 times faster than the conventional well-known program, which took about 261 seconds on average.

TABLE 6
The time taken to form fastq file after
completion of primer removal (sec)

| | cutadapt | present invention |
|---|---|---|
| sample1 | 238 s | 57 s |
| sample2 | 294 s | 70 s |
| sample3 | 360 s | 63 s |
| sample4 | 234 s | 63 s |
| sample5 | 372 s | 64 s |
| sample6 | 224 s | 58 s |
| sample7 | 236 s | 65 s |
| sample8 | 242 s | 66 s |
| sample9 | 220 s | 58 s |
| sample10 | 234 s | 65 s |
| sample11 | 207 s | 55 s |
| sample12 | 244 s | 76 s |
| sample13 | 248 s | 79 s |
| sample14 | 243 s | 73 s |
| sample15 | 190 s | 50 s |
| sample16 | 258 s | 74 s |
| sample17 | 265 s | 81 s |
| sample18 | 260 s | 76 s |
| sample19 | 274 s | 98 s |
| sample20 | 264 s | 87 s |
| sample21 | 303 s | 98 s |
| sample22 | 241 s | 66 s |
| sample23 | 306 s | 97 s |
| sample24 | 303 s | 95 s |

6-2. Comparison in Residual Read Count After Completion of Primer Removal

For 24 samples, after the primer removal of the method of the present invention and the known program (cutadapt, https://github.com/marcelm/cutadapt) was completed, the number (count) of reads that could be used for analysis was compared. The result showed that the present method had more residual reads that can be analyzed (Table 7, FIG. 5). That is, the conventional well-known program had an average of about 91% of reads left after primer removal, and the present invention had an average of about 95% of reads left after primer removal.

TABLE 7
Number (count) of reads used for analysis
after completion of primer removal

| | | Relative to raw | present | Relative to raw |
|---|---|---|---|---|
| | cutadapt | read (%) | invention | read (%) |
| sample1 | 35036 | 91.409% | 36419 | 95.017% |
| sample2 | 39092 | 91.185% | 40752 | 95.057% |
| sample3 | 34896 | 90.851% | 36813 | 95.842% |
| sample4 | 35406 | 91.062% | 37105 | 95.432% |
| sample5 | 37260 | 91.174% | 38606 | 94.467% |
| sample6 | 33463 | 91.078% | 34673 | 94.371% |
| sample7 | 35823 | 91.781% | 37209 | 95.332% |
| sample8 | 36024 | 91.105% | 37792 | 95.577% |
| sample9 | 33242 | 90.823% | 34830 | 95.161% |
| sample10 | 35851 | 90.198% | 37709 | 94.873% |
| sample11 | 31867 | 90.560% | 33524 | 95.268% |
| sample12 | 36886 | 90.767% | 38559 | 94.884% |
| sample13 | 37757 | 90.655% | 39279 | 94.310% |
| sample14 | 36404 | 90.987% | 37699 | 94.224% |
| sample15 | 28907 | 90.994% | 30398 | 95.687% |
| sample16 | 37932 | 91.257% | 39596 | 95.261% |
| sample17 | 39847 | 90.749% | 41924 | 95.479% |
| sample18 | 37623 | 90.327% | 39793 | 95.537% |
| sample19 | 41852 | 90.481% | 44252 | 95.670% |
| sample20 | 40095 | 91.229% | 41844 | 95.208% |
| sample21 | 45740 | 91.001% | 48043 | 95.583% |
| sample22 | 36428 | 90.984% | 38079 | 95.107% |
| sample23 | 45304 | 90.688% | 47785 | 95.654% |
| sample24 | 44659 | 90.989% | 46869 | 95.491% |

6-3. Comparison in Primer Removal Accuracy

A read classified as having completed primer removal in the well-known primer removal program (cutadapt) and a read classified as having completed primer removal in the method of the present invention were mapped to the reference gene (GrCh37/hg19). The result showed that the well-known program failed to accurately remove the primer sequence (FIG. 6).

Although specific configurations of the present invention have been described in detail, those skilled in the art will appreciate that this description is provided to set forth preferred embodiments for illustrative purposes and should not be construed as limiting the scope of the present invention. Therefore, the substantial scope of the present invention is defined by the accompanying claims and equivalents thereto.

### INDUSTRIAL APPLICABILITY

The method of increasing efficiency of read data analysis in next-generation sequencing (NGS) based primer removal according to the present invention has a high speed of data analysis and can accurately remove only primer sequences, thereby being useful for improving efficiency and accuracy of read data analysis.

1. A method of increasing the accuracy of analysis of read data through primer removal in next-generation sequencing (NGS), comprising:
(a) acquiring a read through amplicon-based next-generation sequencing;
(b) analyzing a primer sequence and the read sequence to determine the primer sequence in the read sequence; and
(c) removing the determined primer sequence.

2. The method according to claim 1, wherein step (b) comprises:
(i) extracting a read sequence completely matching a primer sequence from the read sequence;
(ii) extracting a read sequence matching the primer sequence at a reference error value (%) from the read sequence not extracted in step (i); and
(iii) determining primer sequence information of the read based on primer sequence information inside the read from the primer sequence and the read sequence not extracted in step (ii).

3. The method according to claim 1, wherein the read sequence of step (b) is characterized in that a 5′ portion is removed in an amount of 1 to 65%.

4. The method according to claim 2, wherein the sequence comparison in step (i) is characterized by comparing the primer sequence with 20 bp to 70 bp of the 5′ portion of the read sequence.

5. The method according to claim 2, wherein the sequence comparison in step (i) is carried out using an Aho-Corasick algorithm.

6. The method according to claim 2, wherein the reference error value (%) in step (ii) is 0.1% to 10%.

7. The method according to claim 2, wherein the primer sequence information inside the read in step (iii) is information corresponding to the primer sequence of another read present inside the read sequence.

8. The method according to claim 1, wherein the determining the primer sequence in step (b) comprises determining and saving read information and primer information when the primers of the read are forward (5′) and reverse (3′) primers, respectively, and correspond (match) to each other, based on the result of sequencing of the first and second reads.

9. The method according to claim 1, further comprising determining and reporting the ratio of the read in which the primer sequence is determined from the entire read sequence in step (b) to the read in which the primer sequence is not determined therefrom.

10. The method according to claim 1, further comprising reporting the presence or absence of data abnormalities through an amplicon production result wherein, when the next-generation sequencing method is based on amplicon.

11. The method according to claim 10, wherein the amplicon production result is obtained by comparing the amplicon production result predicted based on the primer matching result of an experimental sample with the amplicon production result of the experimental sample compared to an actual control sample.

12. A computer system comprising a computer-readable medium encoded with a plurality of instructions for controlling a computing system to perform primer sequence removal in next-generation sequencing (NGS),
wherein the computer system comprises:
(a) acquiring a read through amplicon-based next-generation sequencing;
(b) analyzing a primer sequence and the read sequence to determine the primer sequence in the read sequence; and
(c) removing the determined primer sequence.

13. The computer system according to claim 12, wherein step (b) comprises:
(i) extracting a read sequence completely matching a primer sequence from the read sequence;
(ii) extracting a read sequence matching the primer sequence at a reference error value (%) from the read sequence not extracted in step (i); and
(iii) determining primer sequence information of the read based on primer sequence information inside the read from the primer sequence and the read sequence not extracted in step (ii).


SEQUENCE LISTING (2 sequences)

SEQ ID NO: 1 - DNA, 138 bp, Artificial Sequence
  Synthetic construct
ttgctttaggaaatactaaggaacttcatgaaacagacttgacttgtgtaaacgaacccattttcaagaactctaccatggttttatatggagacacaggtgataaacaagcaacccaagtgtcaattaaaaaagatt

SEQ ID NO: 2 - DNA, 138 bp, Artificial Sequence
  Synthetic construct
ttgctttaggaaagcctaaggaacttcatgaaacagacttgacttgtgtaaacgaacccattttcaagaactctaccatggttttatatggagacacaggtgataaacaagcaacccaagtgtcaattaaaaaagatt